(19) United States
(12) Patent Application Publication
(10) Pub. No.: US 2022/0061839 A1
(43) Pub. Date: Mar. 3, 2022

(54) Needle Containment Assistant Device for Laparoscopic Surgery

(71) Applicant: Timothy Hardy, Chesapeake, VA (US)

(72) Inventor: Timothy Hardy, Chesapeake, VA (US)

(21) Appl. No.: 17/446,229

(22) Filed: Aug. 27, 2021

Related U.S. Application Data
(60) Provisional application No. 62/706,604, filed on Aug. 27, 2020.

(51) Int. Cl. A61B 17/06; A61B 17/04

(52) U.S. Cl. CPC A61B 17/06161; A61B 2017/00336; A61B 17/0469

(57) ABSTRACT

A Needle Containment Assistant Device for Robot surgeries. The device has an inner tube, an outer sheath, over the inner tube, and placed over the inner tube such that the inner tube can move relative to the outer sheath, a securing device, which allows securing the inner tube to the outer sheath, when the inner tube is at a desired location relative to the outer sheath wherein, when secured by the securing, the inner tube becomes in movable relative to the outer sheath. A needle holding device, has a central hole, where the inner tube passes through the central hole, the needle holding device having a cup shaped needle holding portion, adapted for holding needles therein.

This application claims priority from Provisional 62/706,604, filed Aug. 27, 2020, the entire contents of which are herewith incorporated by reference.

### BACKGROUND

The full visualization of the uterus is an important feature to allow a successful gynecological laparoscopic surgery.

A uterine manipulator allows the operator to flex and rotate the uterus so that it can be brought into an optimal position for the surgery.

### SUMMARY OF THE INVENTION

The inventor recognized, however, that there are a number of drawbacks with the current systems.

An embodiment describes a device for surgical operations, that uses an inner tube and an outer sheath, over the inner tube, and placed and configured such thover the inner tube such that the inner tube can move relative to the outer sheath. A needle holding cup is placed at an end of the outer sheath in a way that the inner tube can extend through the cup. having a cup shaped needle holding portion, adapted for holding needles therein, and having a removable lid, the lid having at least one opening through which needles can be inserted and removed, and having a central opening through which the inner tube passes.

The inner tube can be secured to the outer sheath, when the inner tube is at a desired location relative to the outer sheath.

In an embodiment, the device is used for laparoscopic surgery, and a device with the hooks is attached to the end of the tube, enabling pulling tight of tissue as necessary. The device is inserted with sutures in the cup, which have their threads extending, and can be removed by pulling on the thread. After the needles have been used, they are inserted back into the cup, for removal from the body. placed at an end of the outer sheath in a way that the inner tube can extend through the cup

### BRIEF DESCRIPTION OF THE DRAWINGS

In the Drawings:

FIG. 1 shows an embodiment of the manipulator device.

FIG. 2 shows a needle holder part;

FIG. 3 shows the needle holder part attached to the tube;

FIG. 4 illustrates the needle holding cup;

FIG. 5A shows the attachment module with its arms unfolded;

FIG. 5B shows the attachment module with its arms folded in a position for insertion;

FIG. 6 shows a stretchable plastic hook which can be used with the present system.

### DETAILED DESCRIPTION

The present application describes a uterine manipulator with a needle containment device, used for laparoscopic operations. An embodiment describes using this for a laparoscopic hysterectomy, however, this system can be used for any laparoscopic operation. An embodiment facilitates closure of the Apex of the vagina with sutures and facilitates keeping track of needles in the abdomen.

0-Vicryl sutures can be placed in the cervical stroma at the 3- and 9-o'clock positions to aid in placement of the uterine manipulator and other operative devices.

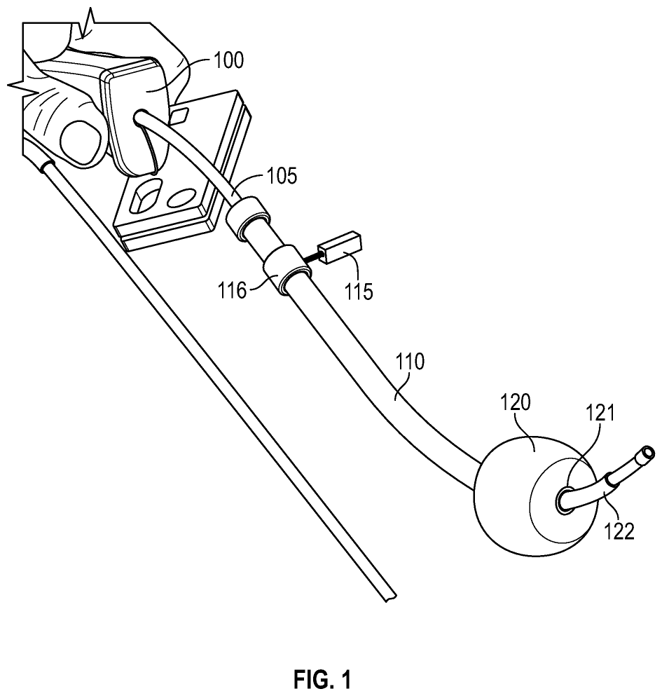

FIG. 1 shows a handle 100 that includes a tube 105 which can be 2-10 mm, in an embodiment 5 mm. There are multiple curves in the tube 105. A plastic sheath 110 covers the tube 105. The tube as covered becomes an inner tube 105 that can be moved relative to the sheath 110. When the tube is in any desired location, the tube 105 can be locked in place relative to the sheath 110 by a set screw 115 which is located in a collar that surrounds the sheath 110 and includes a part that presses in to hold pressure between the sheath 110 and the collar 116. The end part of the device has a cup 120 to fit over the cervix. The cup 120 is located at the end of the sheath 110. In a typical embodiment, the cup 120 is sized fit over the cervix in order to facilitate the surgery. The cup has a hole 121, through which the end 122 of the tube 105 can extend and retract as the tube 105 is moved relative to the sheath 110.

Once the extending part 122 of the of the tube is set to the proper distance, to reach the right depth within the patient's vagina, the set screw 115 is tightened to lock the tube 105 in place, having a desired amount of extending end 122.

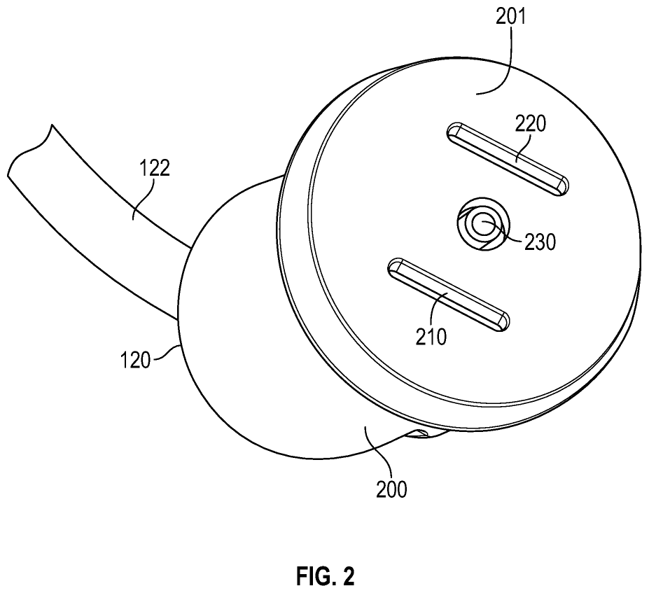

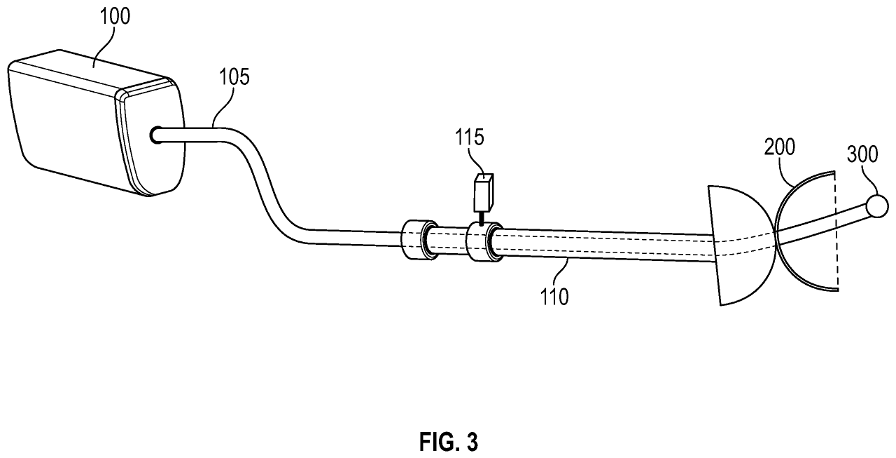

A needle holder part 200, shown in FIGS. 2 and 3, is then attached over the cup 120. The needle holder part 200 has a central hole 230, through which the tube 105 extends. This has the effect of aligning the needle holder part 200 coaxially with and aligned with the cup and the tube, so that the tube 105 extends through the hole in the cup 120, and the hole 230 in the needle holder.

The needle holder part 200 includes first and second slits 210, 220 on its top surface 201, spaced relative to hole 230, through which the tube 105 can pass. The second and second slits 210, 220 are preloaded with sutures, along with hook shaped needles attached to the sutures, where the hook shaped needles are hooked into the slits 210, 220. Any number of sutures can be attached in the slits.

The lid of the cup is also removable via a snap connection, allowing the sutures to be placed inside the inside surface of the cup. For example, 2-3 sutures can be placed inside the cup.

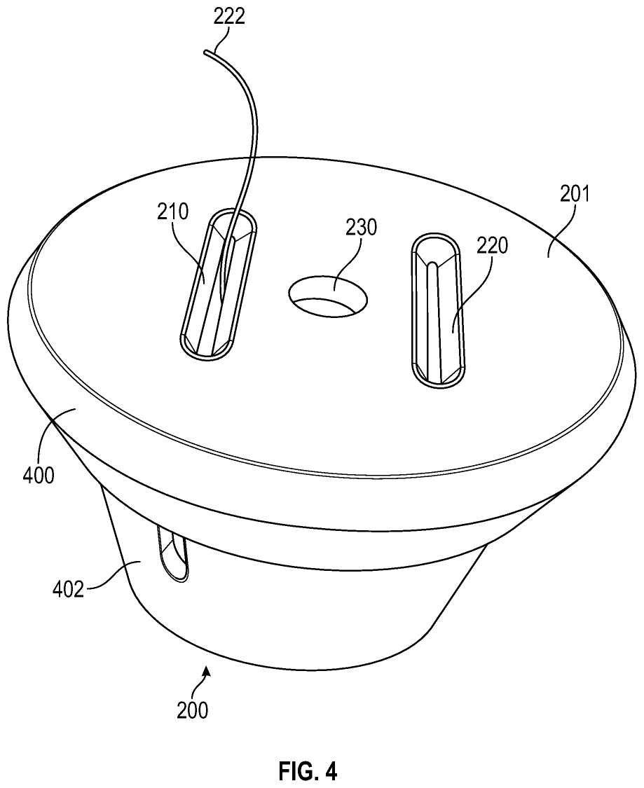

FIG. 4 shows further details of the cup assembly 200, with its needle storage part 402, and its snap on top 400 showing the slots 210 220, and the central hole 230 through which, in operation the tube and extends. The cup assembly 200 is in operation placed on top of the cup 120.

Once assembled in this way, the needle holder and sutures are then placed in the vagina. At this point, sutures have been inserted inside the cup assembly 200, and the suture ends 222 are extending out of the needle holder slots 210, 220.

A robotic device is then used to grab the suture end that is extending out of the needle holder slots 210, 220. The sutures can thus be individually removed while in the vagina and used for suturing using the robot.

FIG. 3 illustrates how an end part 300 of the tube extends at the end of the needle holder 200, when the needle holder 200 is located attached to the top of the cup 120.

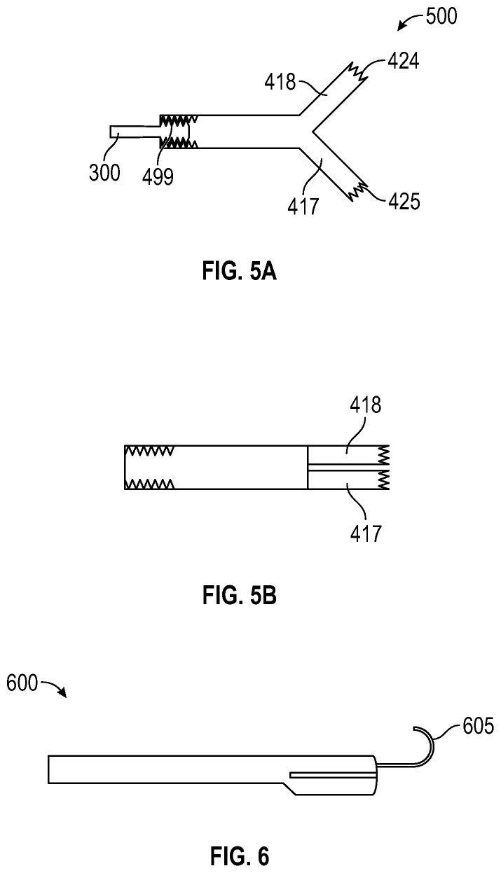

FIG. 5A shows an openable arm piece 500, e.g., formed of plastic, that that in operation is then placed over the extending tube end 300. The plastic piece is shown with a plurality of V grooves 424, 425 on its distal end. The arm piece is inserted e.g. through a laparoscopic port, in its folded position so that the arms 417,418 are folded as shown in FIG. 5B. Then, the device is unfolded to the position shown in FIG. 5A. The device 500 is attached onto the end 300 of the tube 105 either before or after unfolding.

In an embodiment, the tube 105 can have a male screw thread mating with a female thread 499 on the inside of the arm piece 500.

The arm piece can be used to hold the C hooks in place, as described and shown in FIG. 6.

FIG. 5a shows the piece in the open position, in which hooks, as shown in FIG. 6, can be attached to the V-shaped grooves. FIG. 5B shows the piece 500 in its closed position, as it is inserted through a laparoscopic port.

Stretchable elastic pieces 600 have the C hooks 605 attached that can then be used to grab the tissue, and hold the tissue tight, while the other end of the needles is held in the V grooves 424, 425. Spring action will hold the tissue tight at this point.

In one embodiment, the two sets of V-Hooks respectively, 424, 425 can hold the hooks.

These items are brought in and out through the laparoscopic ports, from above.

Once the needles with sutures and/or hooks have been used, they are placed in the slits 210, 220 in the holder part 402 of the needle cup, where they are stored. The whole device is removed at this point. Once removed, the needle holder is opened, to expose the needles, which in this way are contained within the needle holder device, and cannot get lost in the abdomen.

This invention can be used with any kind of laparoscopic surgical device.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A device for surgical operations, comprising:
an inner tube;
an outer sheath, over the inner tube, and placed over the inner tube such that the inner tube can move relative to the outer sheath;
a securing device, which allows securing the inner tube to the outer sheath, when the inner tube is at a desired location relative to the outer sheath wherein, when secured by the securing, the inner tube becomes in movable relative to the outer sheath; and
a needle holding device, having a central hole, where the inner tube passes through the central hole, the needle holding device having a cup shaped needle holding portion, adapted for holding needles therein, and having a removable lid, the lid having at least one opening through which needles can be inserted and removed during the surgical operation and while the needle holding device is inserted in a human body.

2. The device as in claim 1, wherein the cup shaped needle holding portion includes at least one suture with a needle therein.

3. The device as in claim 1, wherein the cup shaped needle holding portion includes a removable cap, and where the removable cap includes first and second elongated slits thereon.

4. The device as in claim 3, wherein the cup shaped needle holding portion includes needles attached to sutures, the sutures extending through the slits.

5. The device as in claim 1, further comprising an arm piece, which attaches to an end portion of the inner tube, and allows movement of the arm piece when the securing device is loosened, the arm piece having at least one V-shaped groove adapted to hold a needle.

6. The device as in claim 5, where the arm piece includes first and second arms, which are foldable relative to one another into a flat position through which they can be inserted through a laparoscopic port, and opened to have a Y shape, and a distal end of each of the arms includes V shaped grooves thereon of a type which are adapted to hold needles.

7. The device as in claim 6, where the assembly is folded flat, and inserted through the laparoscopic port, and then opened to have the y-shape.

8. The device as in claim 5, further comprising hooks, with elastic parts, where the hooks hook onto the V groove, to hold tissue in place.

9. The device as in claim 1, wherein the device is used for gynecological laparoscopic surgery, and further comprising a cup device that extends into a woman's vagina.

10. A method of carrying out a laparoscopic surgical operation, comprising:
using a device having an inner tube, an outer sheath, over the inner tube, and placed over the inner tube such that the inner tube can move relative to the outer sheath, a securing device, which allows securing the inner tube to the outer sheath, when the inner tube is at a desired location relative to the outer sheath wherein, when secured by the securing, the inner tube becomes in movable relative to the outer sheath, and has a needle holding device, having a central hole, where the inner tube passes through the central hole, the needle holding device having a cup shaped needle holding portion, adapted for holding needles therein, and having a removable lid; and
inserting needles through a slot in the lid during a surgical operation, and removing needles from the slot in the lid during the surgical operation.

11. The method as in claim 10, further comprising inserting an arm piece in the body cavity, attaching the arm piece to an end portion of the inner tube, and allowing movement of the arm piece when the securing device is loosened, the arm piece having at least one V-shaped groove adapted to hold a needle.

12. The method as in claim 11, where the arm piece includes first and second arms, which are foldable relative to one another into a flat position through which they are inserted into the body cavity through a laparoscopic port, and opened to have a Y shape, and a distal end of each of the arms includes V shaped grooves thereon of a type which are adapted to hold needles.

13. The method as in claim 12, where the assembly is folded flat, and inserted through the laparoscopic port, and then opened to have the y-shape.

14. The method as in claim 10, wherein the device is used for gynecological laparoscopic surgery, and further comprising a cup device that extends into a woman's vagina.
